(19) United States
(12) Patent Application Publication
(10) Pub. No.: US 2007/0270745 A1
(43) Pub. Date: Nov. 22, 2007

(54) Vacuum actuated tissue lifting device

(76) Inventors: Camran Nezhat, Palo Alto, CA (US); Catherine Mohr, Mountain View, CA (US)

Correspondence Address: GLENN PATENT GROUP, 3475 EDISON WAY, SUITE L, MENLO PARK, CA, 94025, US

(21) Appl. No.: 11/437,035

(22) Filed: May 18, 2006

(51) Int. Cl. A61M 5/00

(52) U.S. Cl. 604/115

(57) ABSTRACT

A vacuum-actuated tissue lifting medical device for creating an operative space in a patient for a surgical procedure is disclosed. The device includes a housing having an opening for resting on a tissue surface of a patient. The housing defines an expansion space between the housing and the tissue surface for application of a negative pressure in the expansion space. A penetrator device located within the housing can penetrate into the tissue surface of the patient. A vacuum system is in fluid communication with the housing for creating a negative pressure in the expansion space for advancing the tissue surface onto the penetrator device such that an operative work space is created beneath the tissue surface. A regulator controls the negative pressure in the housing when (i) the negative pressure in the expansion space exceeds a value; (ii) the pressure in the operative work space exceeds a value; or (iii) a combination of (i) and (ii).

### FIELD

This invention relates to a medical device for lifting tissue to create an operative space in which a tissue perforation device can be inserted. More specifically, the invention relates to a medial device that can control the height of the rise of tissue for creation of such operative space.

### BACKGROUND

Laparoscopy is usually performed under general anesthesia; however it can be performed with other types of anesthesia that permit the patient to remain awake. The typical pelvic laparoscopy involves a small (½″ to ¾″) incision in the belly button or lower abdomen. The abdominal cavity is filled with carbon dioxide. Carbon dioxide causes the abdomen to swell which lifts the abdominal wall away from the internal organs, so the doctor has more room to work. Next, a laparoscope (a one-half inch fiber-optic rod with a light source and video camera) is inserted through the belly button. The video camera permits the surgeon to see inside the abdominal area on video monitors located in the operating room. Depending on the reason for the laparoscopy, the physician may perform surgery through the laparoscope by inserting various instruments into the laparoscope while using the video monitor as a guide.

Although carbon dioxide insufflation has provided a suitable means for creating an operative space in the abdominal cavity, significant morbidity and mortality does occur each year by iatrogenic injuries during establishment of pneumoperitoneum prior to surgical procedures. The main source of these injuries is inadvertent perforation of blood vessels or organ structures within the abdominal cavity when the penetration device (needle, trocar or punch biopsy cutting tool) is advanced too far through the abdominal wall piercing the underlying organs that are located adjacent thereto. These injuries are more common with inexperienced surgeons, but can occur even in the most experienced hands. Built-in safety devices exist in the perforation devices themselves, but injuries still occur because of the close proximity of the structures that are intended to be perforated and those to be avoided.

Alternatives to the methodology of application of carbon dioxide include mechanical lifts which are designated to elevate the abdominal wall during the procedure. In some applications, mechanical wall lifting devices are inserted into the abdominal cavity and actuated to physically lift the interior tissue surfaces of the abdominal wall. Mechanical wall lifting devices have significant drawbacks, including adding an operation step, further obstructing an already limited work space, and providing an obstruction for the surgeon or an obstacle around which the surgeon must work. Moreover, such mechanical devices create a “tent-like” work space, which geometrically is smaller that a dome-shaped work space. Further, mechanical devices may apply pressure on internal organs which can lead to further complications for the patient.

Vacuum devices have been proposed as a very preferable means of lifting the abdominal wall for creating an operative space within the abdominal cavity. An example of a patent that teaches such a device is U.S. Pat. No. 4,633,865. A significant drawback of the device disclosed by this patent is that when the abdominal wall is lifted by the application of the vacuum, the internal organs within the abdominal cavity rise concomitantly with the upward movement of the abdominal wall. Consequently, an operative space will not be provided or a very minimal operative space will be provided, increasing the risk of iatrogenic injuries.

U.S. Pat. No. 6,042,539 discloses a vacuum-actuated tissue lifted device as well. The device has a shell with a profile configuration to surround a tissue surface of the patient, a vacuum port located on the shell for applying a vacuum between the shell and the tissue surface, and an air conduit extending through the shell to permit air to pass into the operative space of the patient when vacuum is applied. Significant improvements, however, can be made to such devices, such as better control over the application of the vacuum being applied within the shell and control by the operator over the height of the tissue rise. Moreover, the device of U.S. Pat. No. 6,042,539 provides for gross distortion of tissue while local distortion may be preferred. The embodiments of the present invention address these as well as other needs.

### SUMMARY

In accordance with one aspect of the invention, a vacuum-actuated tissue lifting medical device for creating an operative work space in a patient for a surgical procedure is provided. The device comprises (a) a housing having an opening for resting on a tissue surface of a patient, the housing defining an expansion space between the housing and the tissue surface for application of a negative pressure in the expansion space; (b) a penetrator device located within the housing for penetrating into the tissue surface of the patient; (c) a vacuum system in fluid communication with the housing for creating a negative pressure in the expansion space for advancing the tissue surface onto the penetrator device such that an operative work space is created beneath the tissue surface; and (d) a device or other means for regulating the negative pressure in the housing when (i) the negative pressure in the expansion space exceeds a value; (ii) the pressure in the operative work space exceeds a value; or (iii) a combination of (i) and (ii). The device or other means for regulating can include a valve in fluid communication with the housing and in fluid communication with the penetrator device. The penetrator device can allow for air to pass into the patient for creating the operative work space. The penetrator device can be in fluid communication with an insufflation gas source for application of a gas into the patient for creating the operative work space.

In some embodiments, means for regulating the negative pressure includes a valve in fluid communication with the insufflation gas source as well as the penetrator device such that the pressure in the operative work space is the insufflation pressure caused by the gas being applied into the operative work space.

In some embodiments, the device can include a penetrator valve for regulating the gas movement into the penetrator device. A conduit can be provided for connecting the penetrator valve to the means for regulating the negative pressure in the housing, such that the conduit is adapted to connect to a gas source.

In some embodiments, the device is for non-gross, local distortion of the abdominal wall.

In some embodiments, the penetrator device is in a stationary position with respect to the housing during the advancement of the tissue surface onto the penetrator device.

In accordance with another aspect, a method of performing a surgical procedure on a patient is provided comprising: (a) positioning a housing having a penetrator device disposed therein over a tissue surface of a patient; (b) applying a vacuum to the housing to create negative pressure in the housing such that the tissue surface is advanced towards the penetrator device; (c) causing the penetrator device to pierce into the tissue surface and into an operative work space; (d) applying a gas from the penetrator device into the operative work space for the expansion of the operative work space; and (e) regulating the negative pressure in the housing based on (i) the pressure in the operative work space; (ii) the pressure in the housing; or (iii) a combination of (i) and (ii).

### BRIEF DESCRIPTION OF THE DRAWINGS

FIG. 1 depicts a cross-sectional view of a controlled-release vacuum apparatus in accordance with one embodiment of the invention positioned over an abdomen of a patient.

FIG. 2 depicts a cross-sectional view of the controlled-release vacuum apparatus wherein a vacuum is produced within the housing lifting the abdominal wall over which the apparatus is placed.

FIG. 3 depicts a cross-sectional view of the controlled-release vacuum apparatus during use in accordance with one embodiment of the invention.

FIG. 4 depicts a cross-sectional view of the controlled-release vacuum apparatus during use in accordance with one embodiment of the invention.

FIG. 5 depicts a cross-sectional view of a controlled-release vacuum apparatus at an equilibrium pressure.

### DESCRIPTION

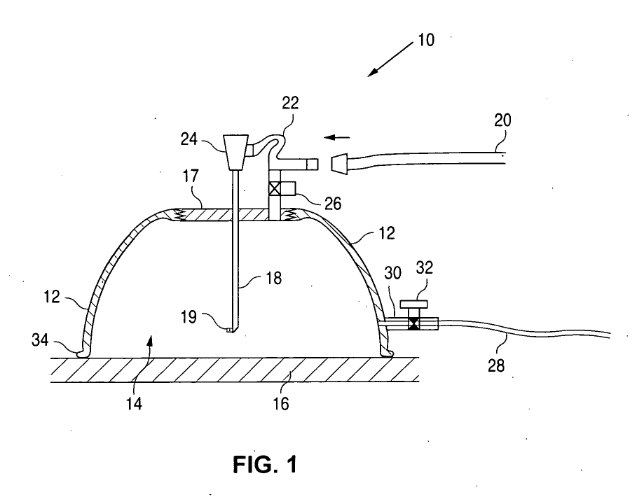

FIG. 1 depicts a cross-sectional view of controlled-release vacuum apparatus 10. FIG. 1 depicts controlled-release vacuum apparatus 10 prior to the penetration of a penetrator device 18 into the abdominal wall of a human or veterinary patient. Controlled-release vacuum apparatus 10 includes an air-tight housing 12 having an opening 14 and a housing pass-through 17. Air tight is indented to include allowing no air into housing 12 or a minimal amount of air such that the desired application of vacuum is not interfered with. Housing 12 can be dome shaped or generally domed shaped and can be made of one piece or half-shells capable of being sealable mated with each other. Housing 12 provides for an expansion space, in which pressure can be applied between housing 12 and a tissue 16 of the patient, such as over the abdomen. Opening 14 is sized to allow elevation of abdominal wall 16 away from the underlying organs or vascular structures of a patient. Opening 14 should allow for local distortion of the abdominal wall as opposed to gross distortion provided by devices of larger size. Housing 12 can further include housing seal 34 along the opening 14 of housing 12 to prevent or minimize pressure or vacuum loss. Housing 12 may be sized to accommodate adults, children and infants of different sizes and/or body mass indexes. The diameter of housing 12 in contact with an abdomen of an adult ranges from about 3 inches to about 8 inches and for pediatric patients from about 1.5 inches to about 3 inches. Due to the size of housing 12, opening 14 is sized such that only a small portion of the abdominal wall 16 is raised for penetration purposes such that no gross distortion of the underlying organs occurs. In some embodiments, only a small distortion of underlying organs may occur. Housing 12 is sized to produce the optimum abdominal wall distortion to allow creation of pneumoperitoneum.

Controlled-release vacuum apparatus 10 additionally includes penetrator device 18 secured to controlled-release vacuum apparatus 10 through housing pass-through or port 17. Penetrator device 18 should be capable of being removed from housing 12 while penetrator device 18 is inserted in a patient. The seal and fixation of penetrator device 18 can be done by means of an O-ring, a solid penetratable elastomeric member or other means known in the art for such sealing purposes. In one embodiment, penetrator device 18 is stationary (for example, with respect to housing 12) such that it does not slide up or down with respect to housing 12 while abdominal wall 16 is pulled up by vacuum to be penetrated by penetrator device 18. Penetrator device 18 can include a veress needle, trocar, or any other suitable device designed to penetrate, to cut tissue, allow passage of gas through a hollow bore, deliver a drug, scope the internal cavities, or for any other diagnostic or treatment purposes. For example, penetrator device can be a punch-biopsy tool. Gas hose 20 can be detachably connected to device 10 for ease of operation and maneuvering of device 10. When attached, the medical gas insufflation source (not shown) will be in fluid communication via a coupler tubing 22 with the interior of housing 12 through a pressure relief valve 26 and with penetrator device 18 via a coupler 24. In some embodiments, coupler 24 can be a valve or a stopcock to further control direction of insufflation gas. Valve 24 can be manually opened and closed for allowing air to pass into the operative space of the patient when vacuum is applied. Once the tip of penetrator device 18 is in the tissue, medical gas insufflation source can be in fluid communication with the interior of housing 12 solely through pressure relief valve 26. Insufflation gas is any medical gas including, but not limited to, carbon dioxide, air, or an inert gas and can be pressurized.

Imaging or video device 19 with a light source may also be included at the tip of penetrator device 18 to observe passage of the tip through the underlying tissues. Imaging device 19 should be capable of focusing to within above 0 (e.g., 0.01) to 1 mm of the penetrator tip and be of sufficiently high resolution to discern the characteristics of the tissue being passed by the tip of penetrator device 18. This will give the operator the visual feedback of having entered the peritoneal cavity below. Video device 19 may be a separate device within the bore of penetrator device 18 or an integral device to penetrator device 18. A remote camera with optical transmission through the bore, a fiber optic lens system, direct sensing with charge coupled device (“CCD”) camera or other electronic optical device directly at the penetrator tip may be used.

A vacuum system (not shown) is removably attached and in fluid communication with housing 12 through vacuum hose 28. Vacuum hose attachment 30 controlled by valve, stopcock and/or regulator 32 is coupled to housing 12. Vacuum valve, stopcock and regulator 32 are any standard devices used in the art for such a purpose. The vacuum system through vacuum hose 28 and regulator and/or valve 32 functions to regulate the negative pressure exerted within housing 12.

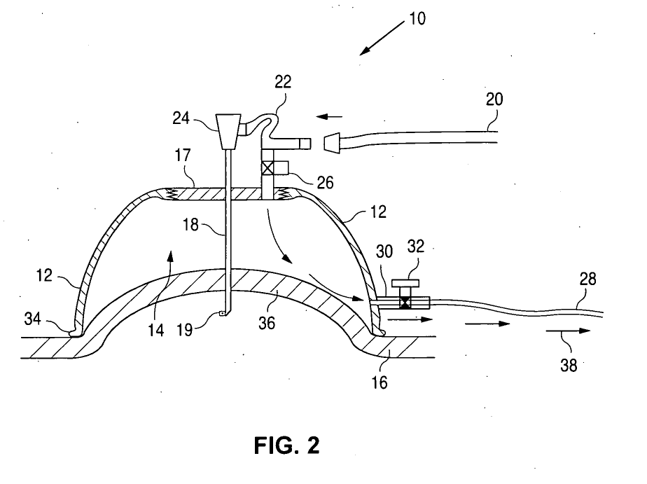

FIG. 2 depicts a cross-sectional view of controlled-release vacuum apparatus 10 with an operating vacuum system attached. A vacuum can be produced within housing 12 positioned over the abdomen of a patient. Valve 24 can be at an open position to allow air to pass into the operative space of the patient when vacuum is applied. Vacuum system through vacuum hose 28 coupled to housing 12 via vacuum hose attachment 30 can be activated to pull air out of housing 12, as depicted by heavy arrows 38, and forming extended abdominal wall “bubble” 36 beneath housing 12. With application of vacuum, extended abdominal wall bubble 36 is produced which elevates abdominal wall 16 away from underlying organs and vascular structures. In some embodiments, vacuum is applied and then terminated such that a generally constant pressure is maintained inside housing 12. In some embodiments, application of negative pressure can be applied at a constant rate for a selected duration of time or adjusted such that pressure inside housing 12 is maintained at a generally same level or is adjusted according to the needs of the patient or the surgeon. In some embodiments, the pressure within the housing can be regulated with a certain degree of accuracy for controlling the safety of the procedure.

As extended abdominal wall bubble 36 is elevated due to negative pressure, penetrator device 18, maintained at a stationary position above the abdominal wall, begins to penetrate into the abdominal wall tissue. The vacuum can produce a selected amount of negative pressure inside the abdominal cavity produced by the local body wall distortion.

As shown in FIG. 2, valve 24 of penetrator device 18 is open to room air. As the tip of penetrator device 18 passes through the peritoneum, the peritoneal cavity will be in fluid communication with room air. As extended abdominal wall bubble 36 is formed by applying negative pressure 38 at vacuum hose attachment 30, creating slight negative pressure inside housing 12, room air will be drawn into extended abdominal wall bubble 36 to allow the underlying organs and vascular structure to fall away from the interior of abdominal wall bubble 36. At this stage, the internal abdominal pressure is the same as the ambient atmospheric pressure.

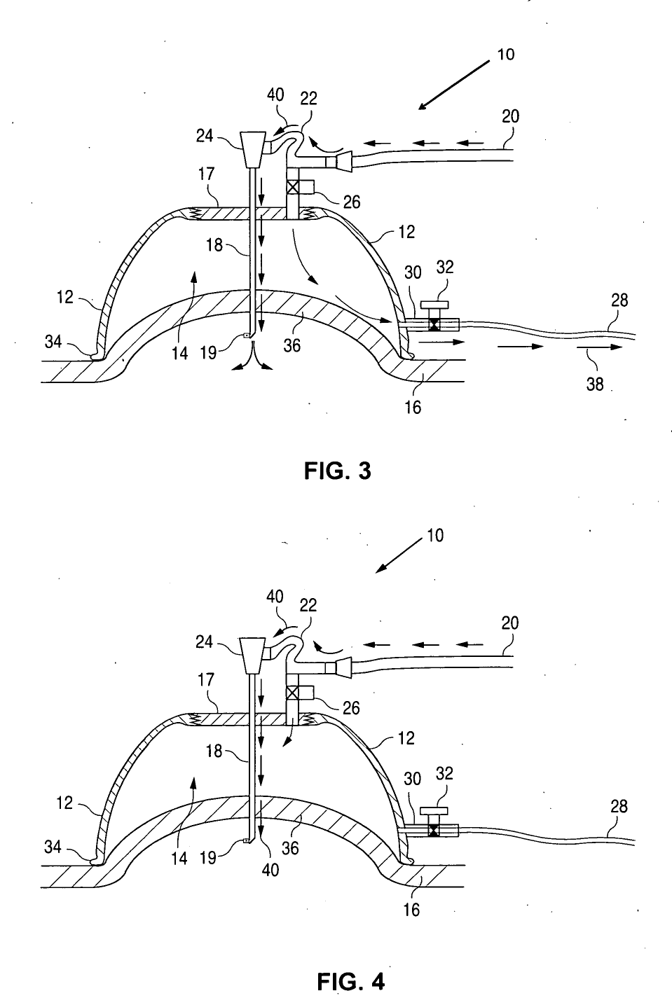

As illustrated in FIG. 3 gas insufflation hose 20 is attached to the controlled-release vacuum apparatus 10. The vacuum system, through vacuum hose 28, continues to hold pressure within housing 12. Gas hose 20, at one end, is in fluid communication with a medical gas insufflation source (not shown) and the other end in fluid communication with the interior of the abdominal cavity through the penetrator device 18. Medical gas 40, such a carbon dioxide or an inert gas is introduced through the penetrator device 18 into the peritoneal cavity to further facilitate separation between abdominal wall 16 and the underlying organs and vascular structures and to create a suitable operative space, such as for laparoscopy.

As one of ordinary skill in the art would readily recognize, a gas insufflation source may be any one of a number of instruments used in medical facilities all over the world. Representative examples include, but are not limited to, the Storz 26012 electronic laparoflator, R. Wolf Co2 Insufflator, Model No.. 2043.5 or the Stryker, Model No. 108621, would all be compatible devices.

The distance of the tissue movement can be controlled directly by the operator through controlling the vacuum. Negative pressure 38 may be controlled by periodic opening of vacuum valve and/or regulator 32. However, with vacuum and/or regulator valve 32 closed, when a vacuum is deactivated the negative pressure 38 within the structure can, in some embodiments, still exists. The vacuum can be initiated or halted through vacuum valve and/or regulator 32, however vacuum valve and/or regulator 32 are not able to create an equilibrium pressure between insufflation gas and vacuum. The vacuum only dissipates if ambient air or other gases are allowed into the structure through the valve or by causing the seal around the bottom edge of housing 12 to leak. In an optimum situation, housing 12 pressure and the insufflation will be controlled such that a surgeon or other medical professional would be confident that the pressure would be easily dissipated once optimum insufflation pressure has been reached and would not exceed a safe level. Insufflation pressure is the pressure as measured within the operative workspace such as the peritoneal cavity.

FIG. 4 depicts controlled-release vacuum apparatus 10 wherein a vacuum 38 is produced within housing 12 and medical gas 40 is applied through gas hose 20 into the operative workspace such as the peritoneal cavity to establish pneumoperitoneum. It is desirable to completely or intermittently release the negative pressure within housing 12 once abdominal wall bubble 36 has been fully insufflated and/or in the instance where vacuum hose 28 has been improperly attached to an unregulated vacuum source because of the adverse effects, such as bruising, that could result.

Pressure within the housing 12 can be relieved if the insufflation pressure rises to a level which is greater than the preset pressure relief valve's release indication. In one embodiment, the measured pressure is 12 mm Hg which is an indication that the patient's abdomen is fully inflated. At this point, pressure relief valve 26 overcomes a pressure relief mechanism (for example, a spring loaded valve) and insufflation gas enters the housing, releasing the vacuum and allowing penetrator device 18 to be removed easily. In some embodiments, pressure relief valve 26 can be held open by constant pressure on the insufflation side.

Pressure within the housing 12 can also be relieved if the negative pressure inside housing 12 exceeds optimum values, thus making the procedure difficult for the practitioner and possibly bruising the patient. The undesirable increase in the housing pressure can be caused by both user error and machine or mechanical failure. In some embodiments, it is preferred that pressure within the housing to be maintained between 50 and 250 mm Hg. Anything above 250 mm Hg could be considered undesirable and potentially dangerous requiring activation of pressure relief valve 26 caused by the pressure differential. Once the differential pressure across pressure relief valve 26 has exceeded the optimum pressure limits because of excess negative pressure inside housing 12, preset pressure-relief valve or gauge 26 that is coupled to coupler tubing 22 between the medical gas insufflation source and housing pass-through 17 is activated, for example intermittently, from housing 12 side releasing insufflation gas 40 into housing 12 to decrease the negative pressure 38 within housing 12. Once the negative pressure is decreased by the release of gas into housing 12, pressure relief valve 26 will close as the vacuum pulling it open decreases. It will behave as a regulator in this orientation.

Different pressures from the positive pressure side (insufflation) or the negative pressure side (vacuum) of the pressure relief valve may be set to overcome the same spring pressure of the pressure relief valve by the effect of the presenting different active areas on the face of the valve mechanism of the vacuum or the positive pressure sides of the valve.

Optimum pressure is subject to many variables, such as the size of the patient, the size of housing 12, and the size and sharpness of penetrator device 18. However, insufflation pressure thresholds which should cause deactivation of penetrator device 18 and vacuum limits which have the potential to cause bruising are common across a wide variety of applications, so these values may be set in preset pressure relief valve 26 for each size of the housing or “bell.”

The determination of optimum pressure for any given procedure may be done manually by the surgeon or other medical practitioner using vacuum valve and/or regulator 32. In some embodiments, the device 10 should allow for easy exchange of regulator 32 that meets the specific need of the particular patient. The pressure relief system provides automatic deactivation of the device once the abdomen is sufficiently insufflated and a backup safety system in event of vacuum valve, regulator or vacuum system malfunction. This pressure relief system may include the use of a CPU to control both the vacuum system and the medical gas insufflation source or the threshold at which the pressure relief valve is opened.

In another embodiment, both the vacuum system and the medical gas insufflation source may be programmed by a user to respond to commands from the CPU to regulate the pressure in an optimal manner for penetration with penetrator device 18. Once negative pressure 38 reaches a predetermined negative pressure limit within housing 12, preset pressure relief gauge 26 will respond to commands from the CPU and release insufflation gas 40 into housing 12.

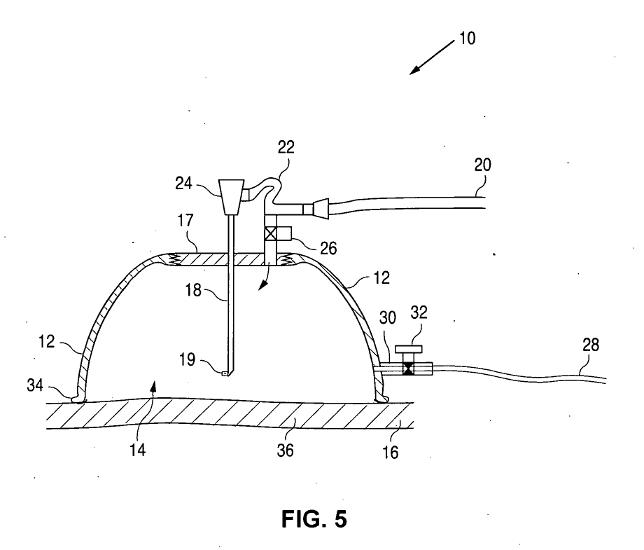

As shown in FIG. 5, once the insufflation pressure has reached the preset pressure, preset pressure-relief gauge 26 releases insufflation gas 42 into housing 12 to equalize the pressure within housing 12. After preset pressure-relief gauge 26 releases insufflation gas 42 into housing 12, abdominal wall bubble 36 deflates significantly. The insufflation gas flow is sufficiently large so as to overcome the flow out through the vacuum system. In this state, vacuum valve and/or regulator 32 may be closed and there will be no negative pressure or vacuum 38 flowing. Once the pressure in housing 12 has reached ambient pressure, seal 34 around opening 14 the perimeter of housing 12 is released and any additional flow from the medical gas insufflation source will be released at the perimeter of the housing 12. Thus, after preset pressure-relief gauge 26 released insufflation gas 40 into housing 12, abdominal wall bubble 36 has almost returned to its normal state.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A vacuum-actuated tissue lifting medical device for creating an operative work space in a patient for a surgical procedure, comprising:
(a) a housing having an opening for resting on a tissue surface of a patient, the housing defining an expansion space between the housing and the tissue surface for application of a negative pressure in the expansion space;
(b) a penetrator device located within the housing for penetrating into the tissue surface of the patient;
(c) a vacuum system in fluid communication with the housing for creating a negative pressure in the expansion space for advancing the tissue surface onto the penetrator device such that an operative work space is created beneath the tissue surface; and
(d) means for regulating the negative pressure in the housing when (i) the negative pressure in the expansion space exceeds a value; (ii) the pressure in the operative work space exceeds a value; or (iii) a combination of (i) and (ii).

2. The device of claim 1, wherein the means for regulating includes a valve-in fluid communication with the housing and in fluid communication with the penetrator device.

3. The device of claim 1, wherein the penetrator device allows for air to pass into the patient for creating the operative work space.

4. The device of claim 1, wherein the penetrator device is in fluid communication with an insufflation gas source for application of a gas into the patient for creating the operative work space.

5. The device of claim 4, wherein the means for regulating includes a valve in fluid communication with the insufflation gas source as well as the penetrator device such that the pressure in the operative work space is the insufflation pressure caused by the gas being applied into the operative work space.

6. The device of claim 1, additionally comprising a penetrator valve for regulating the gas movement into the penetrator device; and a conduit connecting the penetrator valve to the means for regulating the negative pressure in the housing, such that the conduit is adapted to connect to a gas source.

7. The device of claim 1, wherein value is 250 mm Hg for the expansion space and wherein the value is 12 mm Hg for the operative work space.

8. The device of claim 1, wherein the device is for non-gross, local distortion of the abdominal wall.

9. The device of claim 1, wherein the housing comprises
(a) a first port through which the penetrator device is removably connected to the housing;
(b) a second port in communication with the means for regulating the negative pressure;
(c) a passage connecting the penetrator device to the means for regulating the negative pressure; and
(d) a third port for connecting the housing to the vacuum system.

10. The device of claim 1, wherein the penetrator device is in a stationary position with respect to the housing during the advancement of the tissue surface onto the penetrator device.

11. A method of performing a surgical procedure on a patient, comprising:
(a) positioning a housing having a penetrator device disposed therein over a tissue surface of a patient;
(b) applying a vacuum to the housing to create negative pressure in the housing such that the tissue surface is advanced towards the penetrator device;
(c) causing the penetrator device to pierce into the tissue surface and into an operative work space;
(d) applying a gas from the penetrator device into the operative work space for the expansion of the work space; and
(e) regulating the negative pressure in the housing based on (i) the pressure in the operative work space; (ii) the pressure in the housing; or (iii) a combination of (i) and (ii).
